(19) United States
(12) Plant Patent Application Publication
(10) Pub. No.: US 2006/0031973 P1
(43) Pub. Date: Feb. 9, 2006

(54) WALNUT TREE NAMED 'FORDE'

(75) Inventors: Gale McGranahan, Davis, CA (US); Charles Leslie, Davis, CA (US)

Correspondence Address: MORRISON & FOERSTER LLP, 425 MARKET STREET, SAN FRANCISCO, CA, 94105-2482, US

(73) Assignee: UNIVERSITY OF CALIFORNIA, Oakland, CA (US)

(21) Appl. No.: 10/944,092

(22) Filed: Sep. 17, 2004

Related U.S. Application Data
(63) Continuation-in-part of application No. 10/913,249, filed on Aug. 6, 2004, now abandoned.

(51) Int. Cl. A01H 5/00

(52) U.S. Cl. PLT/154

(57) ABSTRACT

A new aad distinct variety of walnut tree denominated ‘Forde’ is descnibed. This new cultivar comes into bearing young, produces well mid-season, and bears a jumbo sized nut with a strong yet easily removed shell. The new tree also shows low susceptibility to walnut blight.

### CROSS-REFERENCE TO RELATED APPLICATrON

The present application is a continuation-in-part of U.S. patnt application Ser. No. 10/913,249, titled WALNUT TREE NAEMED ‘FORDE’, filed on Aug. 6, 2004, which is hereby incorporated by reference in its egtirly.

### FIELD OF THE INVENTION

Botanical/commercial classification: (Juglans regia)/new English walnut varety. Varietal denomintion: cv. Forde.

### BACKGROUND OF TIRE IVNTN

The present invention relates to a new and distinct variety of walnut tree Juglans regia which has been denaominated varietally as ‘Forde,’ and miore particulary to such a walnut tree which has a harvest date three to eight days earlier than the walnut tree variety ‘Chandler’ (U.S. Plant Pat. No. 4,388) and which further produces a walnut that is jumbo in size with light colored kernels and which can be processed in shell or cracked.

It bas long been recognized as desirable to provide walnut trees bearing large crops which are ripe for commercial harvesting and shipment midiseason and exhibit low susceptibility to walnut blight. The tree of the present variety, ‘Forde,’ produces a nut which is similar in some respects to common walnut tree varieties such as ‘Chandler,’ (U.S. Plant Pat. No. 4,388). However, the new variety is ready for harvest up to eight days before ‘Chandler,’ and ten days after the common reference cultivar ‘Payne’ (unpatented).

The new Juglans regia walnut tree of the present invention was created at Davis, Calif. in 1995 by a controlled cross of the cultivars ‘Chico’ and ‘UC 61-25,’ also known as ‘Lara’ (not patented). ‘Porole’ differs from ‘Cbico’ in having larger nuts which are easier to remove from the shell, a later harvested and a more vigorous tree. ‘Forde’ differs from ‘UC61-25’ in having much lighter kernel color, a smoother shell, and unlike ‘UC61-25,’ ‘Forde’ is protogynous. The pedigree is illustrated (FIG. 1).

Seeds from the cross were planted and the resulting 40 trees were carefully observed along with other trees in the walnut breeding program. When they began to bear nuts, data were colleted annually on leafing date, fist poak and last female flower bloom, first, peak and last male bloom, blight severity and yield (Table 1). Nuts were sampled, cracked and data was collected on shell appearance, shell thickness, shell integrity, shell strength, nut weight, kernel weight, percent kernel, ease of kernel removal, kernel color, and percent kernel shrivel (TabIe 2). A single tree was selected from among progeny of this controlled cross based on its superior attributes. This selection was originally designated ‘UC595-26-37,’ and is now designated ‘Forde’ after Harold Forde, a University of California walnut breeder from 1948 to 1978.

The new cultivar ‘Forde’ was originally asexually reproduced by grafting in Davis, Calif., Pirlier, Calif., and Chico, Calif. The new cutitvar of the present invention has been propagated by grafting at Davis, Calif. on ‘Paradox’ hybrid rootstock The distinctive characeristics of the new cultivar have been found to be stable and are transmitted to the new trees when asexually propagated. ‘Forde’ is stable and reproduces true to type.

### SUMMARY OF THE INVENTION

It was found that the new cultivar exhibits the following combination of characteristics:

a) Comes into bearing young, with a good yield for a young tree at age 3;

b) Forms jumbo-sizcd walnuts with little size variation in a given harvest which possess tng well-filled shells and easy to remove light-colored kernels;

a) Can be processed inshelI or cracked;

d) Bears fruit laterally;

e) Yields a walnut crop that can be harvested midseason and prior to ‘Chandler’ (U.S. Plant Pat. No. 4,388);

f) Is protogynous (pollen sheds after female bloom occurs); and

g) exhibits low susceptibility to walnut blight,

### BRIEF DESCRIPTION OF THE TABLES

Table 1 shows comparative tree evaluations.

Table 2 shows nut and kernel traits.

### BRIEF DESCRIPTION OF THE DRAWINGS

FIG. 1—shows the pediee of the ‘Forde’ walnut.

FIG. 2—shows a tree of the ‘Forde’ walnut at seven years.

FIG. 3—shows a near view of the typical current season's stem of the ‘Forde’ walnut.

FIG. 4—shows a near view of the leaves of the ‘Forde’ walnut.

FIG. 5—shows a near view of the bark of the ‘Forde’ walnut.

FIG. 6—shows nuts in the hull of the ‘Forde’ walnut.

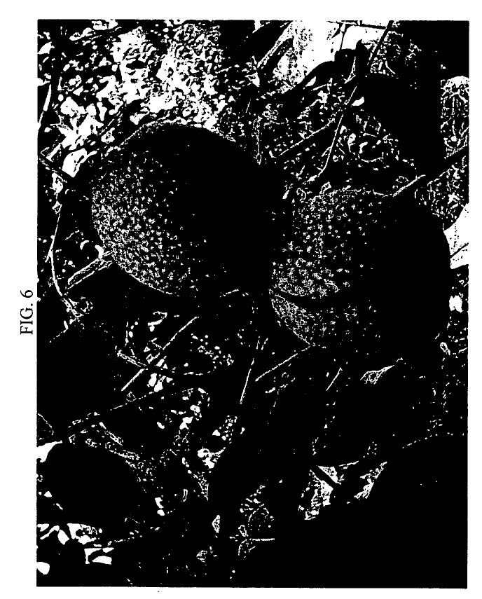

FIG. 7—shows kernels of the ‘Forde’ walnut.

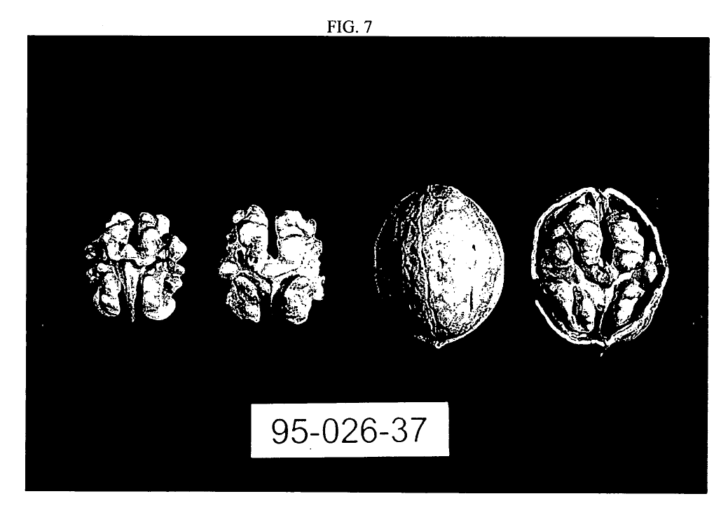

### BOTANICAL DESCRIPTION OF THE PLANT

The description is based on an ungrafted walnut on its own roots and trees propagated by grafting on Paradox rootstock and growing in an orchard at Davis, Calif. Data was collected on the own rooted tree from 1999, age 3 years, to 2003, age 7 years.

In 2001, scionwood from this tree was collected and grafted onto Paradox rootstock for further evaluation in three sites: Davis, Chico and Kearney.

The Munsell Book of Color is used in the identification of color. Also, common color terms are to be accorded their ordinary dictionary significance.

p0 Botanical classification: Juglans regia.

Female parent: UC61-25 (‘Lara’) (unpatented).
Male parent: ‘Chico’ (unpatented).

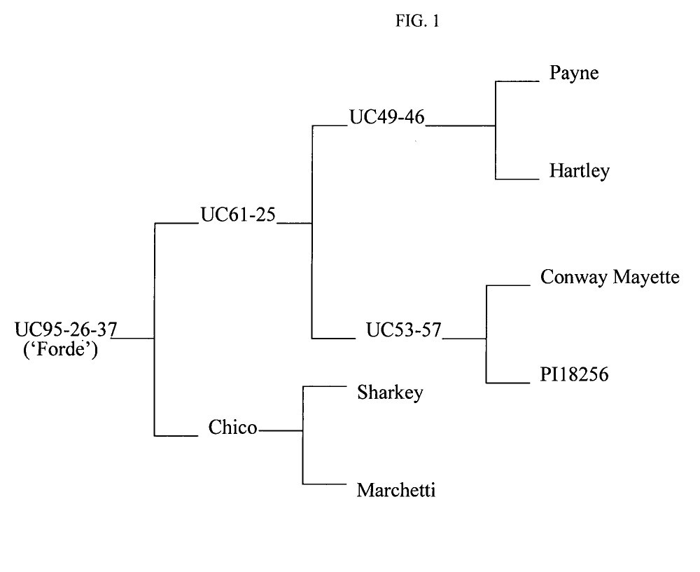

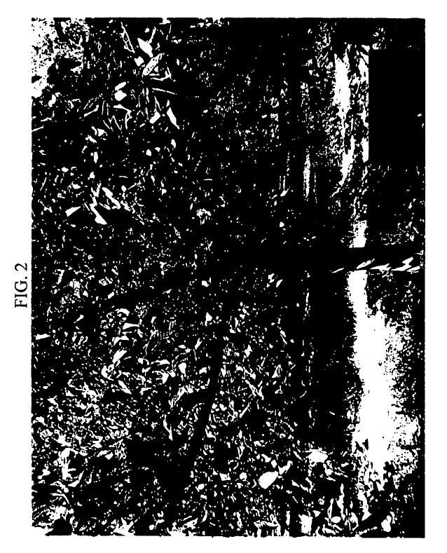

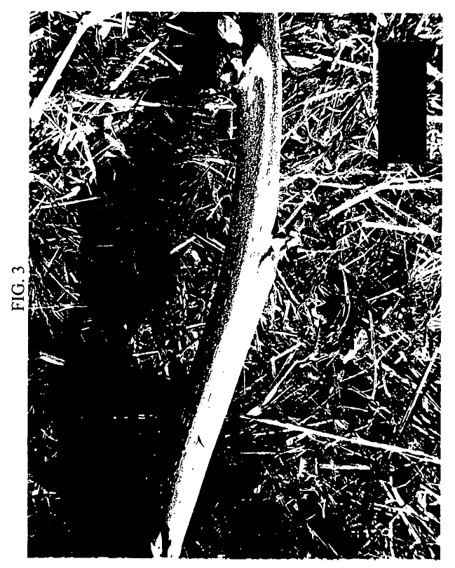

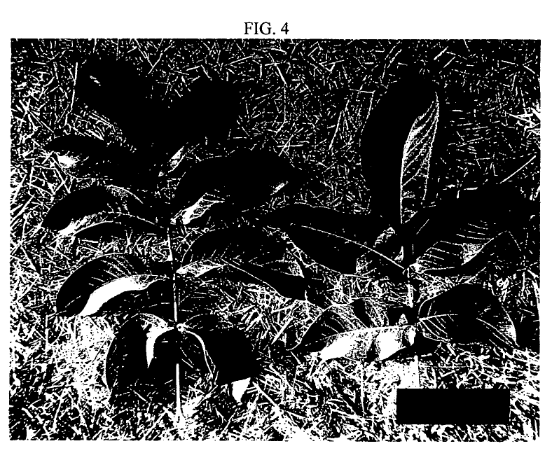

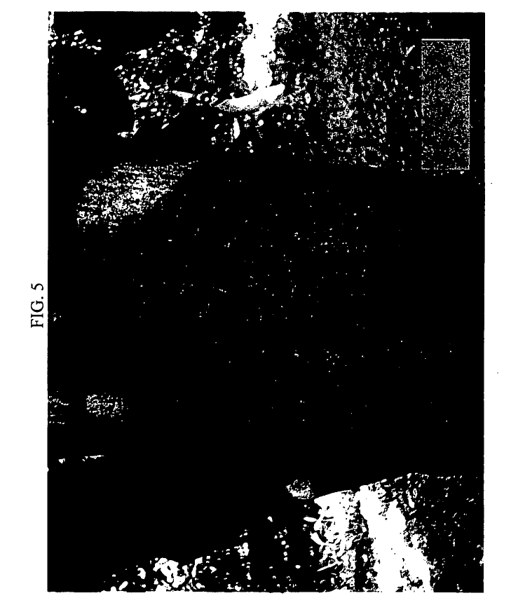

The pedigree is shown (FIG. 1). The growth habit of the tree is illustrated in FIG. 2.
Plant: The growth habit of the tree is illustrated in FIG. 2. This 7 year old tree was approximately 18 feet in height with a canopy diameter of approximately 5.3 meters. The trunk diameter at 30 cm above the ground is approximately 24 cm. The bark (as illustrated) is typical of Juglans regia. The young bark is brown (8.OYR 3.0/5.4) with numerous raised lenticels (FIG. 3). ‘Forde’'s lenticels are oval, measure 2-10 mm by 1-2 mm and are light brown grey 7.0 YR 5.4/1.2 in Munsell's Boook of Color. ‘Forde’'s bark color is grey-yellow (4.4Y 7.2, 3.8) with lighter brown-grey striations (7.0YR 5.4/1.2) (FIG. 5). On one to two year old bark there are about 43 lenticels per 2.5 cm of stem measuring approximately 1.5 cm in diameter, The surface texture of the trunk, branch, leaflets, hull and kernel is smooth. ‘Forde’ has moderate vigor, similar to ‘Chandler’.
Foliage: The dark green foliage is illustrated (FIG. 4) and is typical of Juglans regia. Leaf out during 1999-2003 has occurred on April 1 on the average. For comparative purposes the ‘Payne’ cultivar leafed-out 12 days earlier and the ‘Chandler’ cultivar leafed out 5 days later during the same years. The typical leaf coloration is moderate olive green, 8.2GY 3.2/6.1, on the upper surface and slightly lighter (5.3GY 5.2/9.7) on the lower surface. The leaves are pinnately compound with

5-7 leaflets. The full leaf length is approximately 40.3 cm and the width 29.9 cm. The terminal leaflet averages 15.1 cm in length and 6-3 cm in width. The middle leaflets average 14.9 cm in length ad 5.1 cm in width and the proximal leaflets average 13.7 cm in length and 5.2 cm in width. If 7 leaflets are present the first (proximal) set is smallest averaging 10.1 cm in length and 4.6 cm in width. The leaflets are broadly elliptical and entire. The petiole averages 25.7 cm in length and is 1-2 mm in diameter. The entire rachis including the petiole measures 25.7 cm in length, 24 mm in diameter and the distance to the first leaflet (petiole) is 7 cm. The color is yellow-green 5GY 6/8.
Inflorescence: The tree is relatively precocious; a good yield being noted for a 3-year-old tree. Male flowers (catkins) were not present until age 5. This delay in male maturity is typical of Juglans regia. The catkin's diameter is about 15 mm and yellow-green (5GY 6/8). Catkin length ranges between 7 and 13 cm. From 1999 to 2003, the first female bloom occurred on average on April 7, peak bloom on April 12 and last bloom on April 18. From 1999 to 2003, average male flowering (pollen shedding) began April 15, peaked on April 20 and terminated April 26. In this protogynous tree, pollen shedding covers 15% of the pistillate bloom. A pollenizer would be needed for maximum yield in isolated areas. Both ‘Vina’ (not patented) and ‘UC90-31-10’ (patent applied for, application Ser. No. 10/912,852) would be satisfactory pollenizers. The female flowers are typical of Juglans regia with two flowers per inflorescence borne at both terminal and lateral positions of current season's growth. Approximately 100% of the lateral buds contain inflorescences making yields much greater than trees that only bear flowers terminally. A typical female flower is approximately 5 to 7 mm at anthesis and the floral organs are typical of J. regia. The flower fragrance is typical of J. regia and is not noticeably different than the foliage fragrance. At antithesis the flower itself appears vase shaped with two plumose curved stigmatic arms. The flowers measure 5-7 mm in length and 3-5 mm in diameter and are yellow-green (5GY 6/8) in color. They are borne usually in twos on a 1 cm spike.
Walnuts: The new cultivar harvests on average 3 days before ‘Chandler’ and three weeks after ‘Payne’ but is becoming earlier as the clone ages. In 2003, this new cultivar was ready for harvest on October 3, 7 days before ‘Chandler’ and 12 days after ‘Payne.’ Earlier leafing and harvest dates are typical for walnuts as a clone ages. The new cunltivar has excellent yields of jumbo-sized walnuts. The hull is globose, moderate yellow-green (5GY 5/6), measures 4.7 cm long and 4.1-4.5 mm wide and is 5 mm thick. The oval nutshell is light tan, and measures 40.3 mm in length and 37.3 mm in width. The shell is strong and well sealed and the kernel is easy to remove. The kernel weighs 9.2 g and makes up 53.4% of the total nut weight of 17.2 g. Kernel color is considered excellent and scores mostly in the light to extra light categories of the USDA Standards for Grades of Shelled Walnuts as determined by using the standard Walnut Color Chart for kernels published by te Drieds Fruit Association of Califomia. In addition kernels of Forde scored 54.6 on the Relative Light Index used by Diamond Walnut of Stockton, Calif. The kernels have a shiny surface comparable to that of the ‘Chandler’ walnut. These values are based on 5-year averages of ten walnut samples each year obtained from a young tree. The large plump kernels are approximately 33.6 mm in length and 31.6 mm in width. The kernel is essentially round and splits into halves easily. It is plump in comparson to ‘Chandler’. It is typical of commercial walnuts in terms of flavor and firmness, the latter varying according to the percent moisture after drying.
Hardiess: The immature tee has withstood a temperature of 21° F. in 1998.
Chilling requirement: Trees have not exhibited staggered leafing and bloom. symptoms of lack of chilling since they have been under evaluation, however chilling accumulation (hours under 45° F.) has never been lower than 767 in 1999-2000.

Disease resistance and susceptibility: Susceptibility to walnut blight has been low. No other unusual resistance or susceptibility to insects and diseases has been observed to date.
Usage: The new cultivar of the present invention provides a walnut ultivar that harvests before ‘Chandler’ and has large light and extra light colored kernels that can be used cracked or in shell.

We claim:

1. A new and distinct vaiety of walnut tree substantially as shown and described herein.
